(19) United States
(12) Patent Application Publication
(10) Pub. No.: US 2026/0243576 A1
(43) Pub. Date: Aug. 20, 2026

(54) OPTIMISED MANAGEMENT OF INTERVENTIONS BETWEEN AT LEAST TWO INDIVIDUALS AT THE SAME PLACE OF INTEREST

(71) Applicant: IAVC, Loos (FR)

(72) Inventors: Adrien ROUSSEAUX, Lille (FR); Pierre-Olivier BUSSIÈRE, Bondues (FR)

(21) Appl. No.: 19/161,784

(22) PCT Filed: Mar. 14, 2024
(86) PCT No.: PCT/EP2024/056802

§ 371 Date: Sep. 3, 2025

(30) Foreign Application Priority Data
Mar. 17, 2023 (FR) 2302503

(51) Int. Cl. G01C 21/34

(52) U.S. Cl. CPC G01C 21/3438

(57) ABSTRACT

The present invention relates to a method and a system for synchronising arrivals of at least two individuals at the same place of interest at an arrival time. The first and second individuals are respectively located at first and second starting points at a departure time. The method includes: a) a first geolocation of said first individual at the departure time; b) a first calculation of a first travel time between the first departure point and the place of interest; c) a second geolocation of said second individual at the departure time; d) a second calculation of a second travel time between the second departure point and the place of interest; e) a determination of a departure time.

### TECHNICAL FIELD

The object of the present invention relates to the field of managing interventions (planned or unplanned) between at least two individuals.

One of the objectives of the present invention is to provide a solution for synchronising the respective arrivals of at least two individuals at one and the same place of interest, also called intervention site.

One of the objectives of the present invention relates more particularly to the synchronisation of the respective arrivals of several persons who need to arrive at the same geographical point at the same time or at different times.

The object of the present invention will find advantageous applications in many fields including, in particular, health by enabling interventions to be managed, and whether they are planned or unplanned interventions.

The present invention will, for example, find a particularly advantageous application in the organisation of health professionals for the care of a patient in an emergency situation such as for example the care of a patient suffering a stroke.

It will be understood that the present invention will find applications for the management of other types of patients and will generally apply to the optimised management of all interventions requiring both the transport of a patient to a health institution and the synchronised arrival of at least one health professional at the intervention site.

Of course, the present invention will not apply exclusively to the field of health, but will also find advantageous applications in other fields such as for example car sharing or parcel logistics.

### PRIOR ART

Although this is a major public health issue, the Applicant observes that the management of a patient in an emergency situation is not currently optimised.

The solutions implemented today remain indeed rudimentary.

Traditionally, there are for emergency situations:
systems for geolocation of individuals;
systems for sharing geolocation data;
systems for calculating travel times from a first point to a second point.

By knowing a patient's time and departure point, healthcare professionals can therefore calculate the travel time and of the patient their own travel time at a given time.

To obtain this information (provided they manage to obtain it), healthcare professionals exchange:
by telephone with the Emergency Medical Services (EMS),
between healthcare professionals over the telephone or digital communication tools that are not always secure (usually WhatsApp®).

The information collected (when collected and correct) allows for manual calculation of the time at which the practitioner(s) must leave their location (home or other) to arrive at the intervention site.

On the other hand, it is well known that emergency transport such as mobile emergency service, ambulance or fire service vehicle does not comply with road traffic regulations. To be fair enough, this calculation must be updated regularly depending on where this transport, and therefore the patient, is located. This is currently not possible.

Moreover, it is noted that communication between participants in an emergency situation is often omitted for human and/or organisational reasons.

Let us take the example of a stroke patient.

Stroke is known to be a serious illness that results in a neurological deficit related to vascular-derived brain damage.

The first few hours of care are crucial for the patient.

“Time is brain”: in the event of an ischemic stroke, every minute 2 million neurons disappear, and every 30 minutes that pass represents 15% of additional non-recoverable disabilities.

In some cases, the patient must undergo mechanical thrombectomy at a specialised emergency centre, which involves unlocking the cerebral artery causing the stroke to restore blood circulation and thus limit damage.

In this case, the patient who is taken care of by the emergency services must be transferred as soon as possible from the first healthcare facility to the hospital capable of performing such surgery. For example, in Nord-Pas-De-Calais, only the Lille University Hospital is able to perform mechanical thrombectomies.

Such management must be possible 24 hours a day and 7 days a week.

It therefore requires a dedicated team with specialised personnel; this personnel is therefore most often on call or on standby, and is not necessarily on site.

The Applicant submits here that two thirds of these interventions are performed during on-call or standby periods.

Today, there is no synchronisation solution for the management of these stroke patients.

As soon as the stroke alert is received, the EMS dispatches an emergency vehicle to recover the patient.

This pre-hospital transfer is carried out to a healthcare facility with a neurovascular unit (NVU) or to a telemedicine centre.

By telephone, the EMS notifies healthcare professionals in the healthcare facility that a patient is about to arrive. However, healthcare professionals do not know when and at what moment they need to mobilise, as well as the other human and technical resources in the healthcare facility, to be synchronised with the patient and avoid any loss of time.

The Applicant, on the other hand, submits here that too early mobilisation of these technical and human resources would degrade the efficiency of the care system and the quality of life at work of healthcare professionals, in particular during on-call and standby periods.

Conversely, late mobilisation of these technical and human resources leads to loss of chances for patients.

The patient is then examined and treated.

In France, in 2020, 7189 patients underwent surgery, mechanical thrombectomy. This operation can only be carried out in certain specialised centres, or emergency centres, with an interventional neuroradiology unit (INU).

If the patient has not been taken directly to the hospital with an interventional neuroradiology unit, the patient will then have to be transferred between hospitals.

The teams of the first healthcare institution and the INU then exchange information by telephone to validate the treatment for mechanical thrombectomy.

When this action is confirmed by the INU, the NVU contacts the EMS to expedite a means of transport for the patient. Depending on the time of day, ongoing procedures and other parameters, the time taken to take care of the patient by this means of transport is variable, so that it is impossible to determine in advance when the patient will actually leave from the NVU to the INU.

The NVU team, when they have time, warns that the emergency vehicle is leaving from the NVU to the INU. However, this call is usually forgotten.

Thus, during a pre-hospital transfer and an inter-hospital transfer, it can be stated that healthcare professionals are informed that a patient is about to arrive. However, no one knows exactly when the patient will arrive.

This problem is exacerbated during on-call periods, when the healthcare professionals who must take care of the patient are not on the hospital premises. They are informed that a patient is about to arrive and must do everything they can to arrive before the patient arrives. Yet they do not know when they need to prepare to take care of the patient on time.

Improving the coordination of healthcare professionals, particularly in the stroke sector, is a real expectation.

The Applicant submits that there is no system for synchronising healthcare professionals with patients during their transfer.

In general, there are no systems for synchronising several people in real time so that they go to the place of interest simultaneously, or with a predefined delay time.

### OBJECT OF THE PRESENT INVENTION

The aim of the present invention is to improve the above-described situation.

The present invention aims in particular to remedy at least one of the various technical problems mentioned above by proposing a solution for managing the synchronisation of the arrivals of at least two individuals at one and the same place of interest.

The object of the present invention relates, according to a first aspect, to a method for synchronising the respective arrivals of at least one first and at least one second individual at one and the same place of interest at a time t, referred to as arrival time, for an intervention.

According to the present invention, the first and second individuals are located respectively at first and second starting points at a starting time.

According to the present invention, the second starting point of the at least one second individual is not located in a proximity zone defined around said place of interest. For example, it will be understood that said at least one second individual is at home, at another site, or another room of the same site.

The process is implemented by computer means.

According to the present invention, the method comprises the following steps:
a) a first geolocation of said at least one first individual at the time of departure to determine, using a geolocation beacon, a geographical position of the first starting point;
b) a first calculation by a computer of a first travel time between the first starting point and the place of interest in order to estimate the time of arrival corresponding to the arrival of said at least one first individual at the place of interest;
c) a second geolocation of said at least one second individual at the time of departure to determine a geographical position of the second starting point;
d) a second calculation by said computer of a second travel time between the second starting point and the place of interest;
d) a determination by said processor of a departure time of said at least one second individual as a function of the second travel time and the arrival time;
e) generation and transmission of a warning signal to a communication terminal of said at least one second individual, the warning signal containing intervention information relating to the planning of the intervention at the time of arrival of said at least one first individual at the place of interest and the time of departure to allow said at least one second individual to leave the second starting point in order to arrive synchronously at the place of interest with said at least one first individual.

Thanks to the succession of the above technical steps, it becomes possible to provide a solution for managing the synchronised arrivals of several individuals at the same location.

This is particularly interesting in the field of healthcare to optimise the care of a patient by a healthcare professional in an operating theatre, as is the case, for example, for patients suffering a stroke who have to undergo mechanical thrombectomy.

Advantageously, the departure time is further determined according to a predetermined preparation time.

Advantageously, the method according to the present invention includes:
an interrogation via a central server of an electronic calendar to identify at least one availability of the place of interest;
planning by a processor an intervention at the place of interest by determining, among said at least one availability of the place of interest, the availability of the place of interest closest to the estimated time of arrival.

Advantageously, the planning step comprises a reservation in the electronic calendar of an intervention according to the availability of the place of interest closest to the estimated time of arrival.

Advantageously, the method according to the present invention includes, prior to the first and/or second calculation steps, an interrogation of a remote traffic server to retrieve traffic data between the place of interest and respectively the first and/or second starting points.

Advantageously, the first and/or second calculation steps take into account said traffic data.

Advantageously, the first and/or second calculation steps implement machine learning to calculate the first and/or second travel time respectively.

Advantageously, the method according to the present invention comprises monitoring the position of said at least one first individual on the first route between the first starting point and the place of interest to continuously or periodically re-evaluate the arrival of said at least one first individual at the place of interest.

According to a second aspect, the object of the present invention relates to a computer program which includes instructions adapted for the execution of the steps of the method according to the first aspect of the invention, that being so in particular when the computer program is executed by at least one processor.

Such a computer program can use any programming language, and be in the form of source code, object code, or intermediate code between a source code and an object code, such as in a partially compiled form, or in any other desirable form.

According to a third aspect, the object of the present invention relates to a computer-readable recording medium on which a computer program is recorded comprising instructions for the execution of the steps of the method according to the first aspect of the invention.

On the one hand, the recording medium may be any entity or device capable of storing the program. For example, the medium may include a storage means, such as a ROM memory, a CD-ROM or a ROM memory of the microelectronic circuit type, or a magnetic recording means or a hard disk.

On the other hand, this recording medium may also be a transmissible medium such as an electrical or optical signal, such a signal being able to be conveyed via an electrical or optical cable, by conventional or hertzian radio or by self-directed laser beam or by other means. In particular, the computer program according to the invention may be downloaded from an Internet-type network.

Alternatively, the recording medium may be an integrated circuit in which the computer program is incorporated, the integrated circuit being adapted to execute or to be used in the execution of the considered method.

The object of the present invention relates according to a fourth aspect to a system for synchronising the respective arrivals of at least one first and at least one second individual at the same place of interest at a time t, referred to as arrival time, for an unplanned intervention, According to the present invention, the system comprising means configured to implement the steps of the method according to the first aspect described above.

More particularly, the system comprises:
a) a geolocation module configured to determine a geographical position of the first starting point of said at least one first individual at the time of departure;
b) a computer configured to calculate a first travel time of said at least one first individual between the first starting point and the place of interest in order to estimate the time of arrival corresponding to the arrival of said at least one first individual at the place of interest;
wherein said geolocation module is further configured to determine at the time of departure a geographical position of the second starting point of said at least one second individual; and
wherein the computer is further configured to calculate a second travel time between the second starting point and the place of interest.

According to the present invention, the system further comprises a processor configured to:
determine according to the second travel time and the time of arrival a departure time of said at least one second individual; and
generate and transmit a warning signal to a communication terminal of said at least one second individual, said warning signal containing intervention information relating to the planning of the intervention at the time of arrival (t) said at least one first individual at the place of interest and said departure time to enable said at least one second individual to leave the second departure point in order to arrive at the place of interest in a synchronised manner with said at least one first individual.

Thus, through its various functional and structural technical characteristics described above, healthcare professionals are provided with a reliable and efficient tool to synchronise the arrival of medical staff, in particular on call or on standby, with that of a patient in an emergency situation for optimal care.

It will be understood that the present invention will also be used for applications for carpooling or parcel transport by enabling the synchronisation of the respective arrivals of at least two individuals at the same place of interest.

### BRIEF DESCRIPTION OF THE APPENDED FIGURES

Other features and advantages of the present invention will arise from the description hereinbelow, with reference to the appended figures which illustrate a non-limiting embodiment thereof and wherein:

FIG. 1 schematically shows the treatment pathway of a patient suffering a stroke according to an example of application of the present invention;

FIG. 2 is a flowchart illustrating all the steps of the method according to an example embodiment of the present invention; and

FIG. 3 schematically shows a synchronisation system according to an example embodiment of the present invention.

### DETAILED DESCRIPTION ACCORDING TO AN ADVANTAGEOUS EMBODIMENT

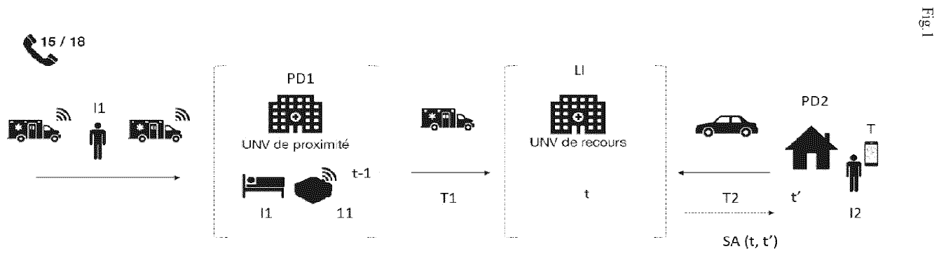

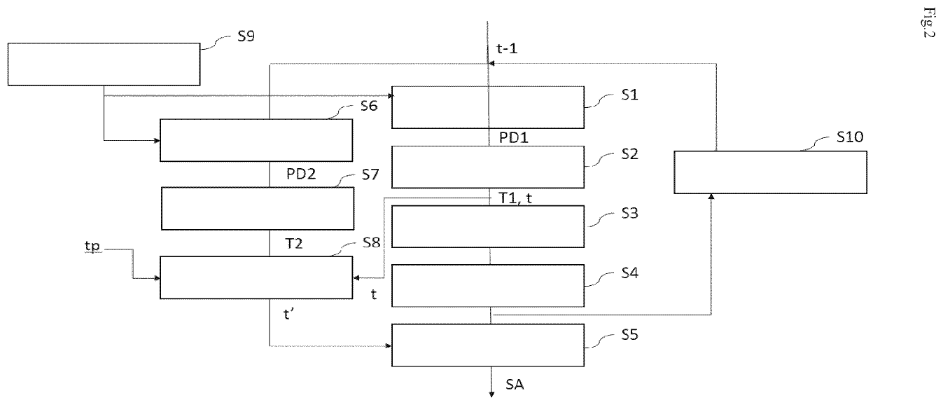

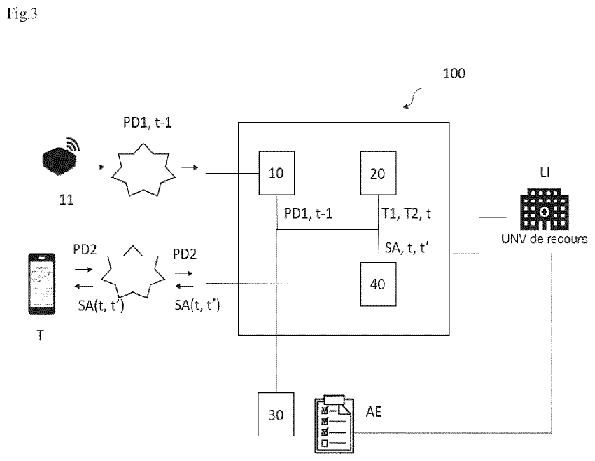

A system for synchronising the respective arrivals of a first and a second individual at the same place of interest for an intervention as well as the method associated with it will now be described in the following with reference jointly to FIGS. 1 to 3.

The example described below relates to the management of a patient suffering a stroke.

This is therefore an unplanned emergency response.

It will be understood here that this is an example of an application among other possible ones which has no limiting character to the present invention, which may apply to other fields.

As explained in the preamble, the management of a patient suffering a stroke is complex and involves a large number of actors, from calling the emergency services (via an emergency number such as “15” or “18”) to discharge from hospital.

When stroke occurs, it is an absolute emergency due to the narrow therapeutic window (up to 6 hours for mechanical thrombectomy).

All healthcare professionals involved in this care chain must therefore interact in a coordinated manner to prevent delays from accumulating and being detrimental to the chances of recovery of the patient.

To date, there is no automated synchronisation solution.

Synchronising the arrival of the practitioner I2 who will perform the thrombectomy with that of the patient I1 suffering the stroke at an intervention site LI is one of the objectives of the present invention.

Such an objective is achieved by the invention described above which provides for a dedicated system 100 for synchronising the respective arrivals of at least two individuals I1 and I2 at the same location LI.

It will be understood here that the present invention may include several persons such as for example several practitioners I2 intervening on the same patient.

In the example described here, the patient I1 suffers a stroke.

As shown in FIG. 1, the first emergency services are notified via an emergency number of the type “15” or “18”. Once they arrive on site, the emergency department performs an initial diagnosis: the I1 patient is then referred to a neurovascular unit (NVU) via an emergency vehicle of the ambulance type.

Examinations are performed at the NVU and a first treatment may already be administered.

In some cases, patient I1 may be eligible for mechanical thrombectomy.

This operation can only be carried out in certain specialised centres, or emergency centres, with an interventional neuroradiology unit (INU).

This INU unit is hereinafter referred to as LI. Here, the intervention site has the hardware infrastructure to perform mechanical thrombectomy.

In this example, the patient I1 must therefore be transferred between hospitals to this intervention site LI.

It is also desirable to ensure that the patient I1 is taken care of immediately by the practitioner I2 once they arrive at this intervention site LI, so as not to lose any minute in care.

The objective here is therefore to synchronise the arrival of the patient I1 and the arrival of the doctor I2 at the interventional neuroradiology unit INU corresponding to the location LI.

Thus, in the example described herein, once the patient I1 is diagnosed as a victim of a stroke and is considered eligible for mechanical thrombectomy, the caregiver places a geolocation tag 11 on them, which is activated during an initialisation step. Such a tag 11 may for example be attached to the ankle of the patient I1 by the emergency nurse.

Once switched on, the tag 11 regularly transmits its geolocation. This geolocation makes it possible to determine whether the tag leaves a predefined perimeter of the NVU. This marks the start time t−1 of the process and the change of the tag to the active state.

Once the tag is activated, a first geolocation S1 of the patient I1 is provided at the departure time t−1.

This step S1 allows the geolocation module 10 to determine geolocation data of the GPS type provided by the tank which correspond to the geographical position of the first starting point PD1 of the patient, here for example the neurovascular unit NVU.

In the example described herein, the system 100 further comprises a computer 20 which receives these geolocation data from the tag 10 and calculates during a step S2 the first travel time T1 between the first starting point PD1, here the NVU, and the place of interest LI, here the angiography room LI of the NVU, in order to estimate the arrival time t which corresponds to the arrival of the patient I1 in this room LI.

The determination of this arrival time t is assessed.

In the example described herein, the processor 40 may first interrogate during a step S9 a remote traffic server (for example a traffic server of the Google Maps® type or equivalent) to retrieve traffic data between the place of interest and respectively the first starting point PD1. It is understood in this example that the computer 20 here takes into account these retrieved traffic data when calculating S2.

In this example, provision is made to implement machine learning during this calculation to calculate the first travel time T1.

In this example, vision is also made to monitor during a step S10 the position of the patient I1 on the first journey between the first starting point PD1 and the place of interest LI to re-evaluate the time of arrival t continuously or periodically.

This makes it possible to have a reliable value that takes into account the various parameters of the arrival of the patient I1 patient at the location LI (traffic, weather conditions, etc.).

In the example described herein, the system 100 then comprises means for, during a step S3, interrogating via a central server 30 an electronic calendar AE and identifying at least one availability of the place of interest LI.

The availabilities of each NVU unit are in fact managed by an electronic calendar AE which enables the schedule of the unit NVU to be managed. Such a calendar is administered by a person from the department concerned who organises the admission schedule.

This schedule is, for example, presented in the form of a spreadsheet, with each box corresponding to a time slot with, for example, the words “available” or “occupied”.

In this example, this interrogation step S3 consists in passing a pointer over this spreadsheet to extract a list of slots available for use of the block in question, such a list comprising at least one availability of said block LI.

According to the present invention, a step S4 is then provided for planning an intervention in the operating room LI by determining, among the availabilities identified during the previous step, the availability of the place of interest closest to the estimated time of arrival t.

This step S4 also includes a booking in the electronic calendar AE of the most relevant slot. Such a planning step S4 is implemented by a processor 40 configured to optimally plan this planning.

Once the intervention has been planned, the processor 40 will then generate and transmit to a communication terminal T belonging to the practitioner I2 a warning signal of the “push” type for example comprising intervention information relating to the planning of the intervention at the time of arrival t of the patient I1 at the place of interest LI.

In the example described here, it is desirable for the doctor 12 to be present on site at the same time as his patient I1, for obvious reasons.

A stroke can occur any day of the week at any time.

The doctor 12 is often on call, for example at home or elsewhere.

To remedy this situation, the method comprises a second geolocation step S6 which aims to geolocate the practitioner 12 at the departure time t−1, once the tag 11 of the patient I1 is activated.

Such a step S6 then makes it possible to determine a geographical position of the second starting point PD2, for example here the home of the doctor.

The calculator 20 then receives this information and calculates during a second calculation step S7 a second travel time T2 between the second starting point PD2 and the place of interest LI.

In the example described herein, this data is then used during a step S8 during which the processor 40 determines the departure time t′ as a function of the second travel time T2 and the arrival time t to allow the practitioner 12 to leave the second departure point PD2 (their home) in order to arrive synchronously at the place of interest LI with the patient I1.

It will be understood here that this departure time t′ is further determined according to a predetermined preparation time tp of the procedure. This preparation time tp corresponds, for example, to the time required for the medical team to organise and/or prepare the thrombectomy equipment and the unit before the procedure as such.

In this example, the procedure information sent via a push message to the doctor includes this departure time t′.

The practitioner I2 then receives, via the software application installed on their terminal T, the information associated with the departure time t′ to arrive on time at the intervention site LI and thus take care of the patient I1 without delay.

The applicant submits that such a solution is particularly appreciated in the field of health. It solves many problems:

The first problem encountered with traditional solutions is related to geolocation; the geolocation of emergency vehicles is not accessible to all EMSs, and so they do not know where the patient is. Traditionally, when they have vehicle geolocation, the problem is that patients can come from several administrative areas, dependent on different EMSs. There are various systems used by EMSs, but they are not interoperable. An EMS does not have the geolocation of the vehicles of the other EMSs.

One technical problem is therefore to know, with their agreement, where the healthcare professionals who have to go to the healthcare facility are located.

Another problem with traditional solutions is related to timing; at the moment, it is difficult or even impossible to know when the patient leaves for the healthcare facility. The caregiver knows that the patient will arrive for care, but does not know when. This is all the more problematic as the caregiver has to prepare the procedure materials before the patient arrives. The time problem also occurs with the unknown travel time, given the conditions of urgency, possible traffic jams or other unforeseen events on the road cannot be fully taken into account. Finally, another problem encountered with traditional solutions concerns the management of confidentiality; it is in fact essential to secure the exchange of information between healthcare professionals. Professionals are not always equipped with solutions that comply with regulations (GDPR and professional secrecy in particular) and data security.

Too often, they use non-secure solutions to share confidential data with each other. These do not allow, in particular:
strong authentication of healthcare professionals
certified hosting of health data;
the non-reuse of data exchanged for commercial purposes.

All of the following problems are solved in the context of the present invention.

The solution provided by the present invention thus resembles a true automated system coupling a geolocation device and a program that calculates the departure time of the healthcare professional to guarantee the arrival time of the healthcare professional with a time delta predefined in advance on the patient.

This solution can of course handle the synchronised arrivals of more than two individuals at the same location, for example one patient and several doctors.

It should be noted that this technical solution is not only aimed at the health sector. The present invention also makes it possible to synchronise the respective arrivals of several people, for example for carpooling or logistics.

It should be observed that this detailed description concerns a particular example embodiment of the present invention, but that this description in no way applies any limiting nature to the subject matter of the invention; on the contrary, it is intended to remove any inaccuracy or any incorrect interpretation of the following claims.

It should also be observed that the reference signs placed between parentheses in the following claims are in no way limiting; these signs are merely intended to improve the intelligibility and comprehension of the following claims as well as the scope of the protection sought.

1. A method for synchronising the respective arrivals of at least one first and at least one second individual at the same place of interest at an arrival time for an intervention,
wherein said at least one first and at least one second individual are located respectively at first and second starting points at a starting time,
said method implemented by computer means comprising:
a) a first geolocation of said at least one first individual at the time of departure to determine, using a geolocation system, a geographical position of the first starting point;
b) a first calculation by a computer of a first travel time between the first starting point and the place of interest in order to estimate the arrival time corresponding to the arrival of said at least one first individual at the place of interest;
c) a second geolocation of said at least one second individual at the time of departure to determine a geographical position of the second starting point;
d) a second calculation by said computer of a second travel time between the second starting point and the place of interest;
e) a determination by a processor a departure time of said at least one second individual as a function of the second travel time and the arrival time;
f) a generation and transmission a warning signal to a communication terminal of said at least one second individual, said warning signal containing intervention information relating to the planning of the intervention at the time of arrival of said at least one first individual at the place of interest and said departure time to enable said at least one second individual to leave the second departure point in order to arrive synchronously at the place of interest with said at least one first individual.

2. The method according to claim 1, wherein the starting time is further determined during the determination step according to a predetermined preparation time.

3. The method according to claim 1 further comprising:
interrogating via a central server an electronic calendar to identify at least one availability of the place of interest;
planning by said processor an intervention at the place of interest by determining, among said at least one availability of the place of interest, the availability of the place of interest closest to the estimated time of arrival.

4. The method according to claim 1, further comprising, prior to the first calculation step and/or the second calculation step, an interrogation of a remote traffic server to retrieve traffic data between the place of interest and respectively the first starting point and/or second starting point, and wherein the first calculation step and/or the second calculation step takes into consideration said traffic data.

5. The method according to claim 4, wherein the first calculation step and/or second calculation step implements machine learning to calculate the first travel time and/or the second travel time respectively.

6. The method according to claim 4, wherein the planning step comprises a booking in the electronic calendar of an intervention according to the availability of the nearest place of interest to the estimated time of arrival.

7. The method according to claim 1, further comprising monitoring the position of said at least one first individual on the first route between the first starting point and the place of interest to continuously or periodically re-evaluate the arrival of said at least one first individual at the place of interest.

8. (canceled)

9. A non-transitory computer-readable recording medium on which a computer program is recorded comprising instructions for performing the steps of the method according to claim 1.

10. A system for synchronising the respective arrivals of at least one first individual and at least one second individual at the same place of interest at an arrival time for an intervention, wherein said at least one first individual and at least one second individual are located respectively at first starting point and second (PD2) starting points at a starting time, the system comprising:
a) a geolocation module configured to determine, using a geolocation system, a geographical position of the first starting point of said at least one first individual at the starting time;
b) a computer configured to calculate a first travel time of said at least one first individual between the first starting point and the place of interest in order to estimate the arrival time corresponding to the arrival of said at least one first individual at the place of interest
wherein said geolocation module is further configured to determine at the starting time a geographical position of the second starting point of said at least one second individual; and
wherein the calculator is further configured to calculate a second travel time between the second starting point and the place of interest;
said system further comprising a processor configured to:
determine according to the second travel time and the arrival time a departure time of said at least one second individual; and
generate and transmit a warning signal to a communication terminal of said second individual, said warning signal containing intervention information relating to the planning of the intervention at the time of arrival of said at least one first individual at the place of interest and said departure time to enable said at least one second individual to leave the second departure point in order to arrive synchronously at the place of interest with said at least one first individual.

11. (canceled)
